(19) United States
(12) Patent Application Publication
(10) Pub. No.: US 2010/0068233 A1
(43) Pub. Date: Mar. 18, 2010

(54) Modifiable dosage form

(75) Inventors: Mahalaxmi Gita Bangera, Renton, WA (US); Edward S. Boyden, Cambridge, MA (US); Roderick A. Hyde, Redmond, WA (US); Muriel Y. Ishikawa, Livermore, CA (US); Dennis J. Rivet, Portsmouth, VA (US); Elizabeth A. Sweeney, Seattle, WA (US); Lowell L. Wood, JR., Bellevue, WA (US); Victoria Y.H. Wood, Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 12/284,013

(22) Filed: Sep. 16, 2008

(51) Int. Cl. A61K 9/127; A61K 9/00; A61K 9/24; A61K 9/52; A61K 9/14; A61K 9/10

(52) U.S. Cl. 424/400; 424/472; 424/457; 424/490; 424/484; 424/450

(57) ABSTRACT

Provided embodiments include a final dosage form, an article of manufacture, and method. A final dosage form for delivering a medicament to an animal is provided. The final dosage form includes an outer layer. The final dosage form also includes a release element configured in a first medicament-release state and modifiable to a second medicament-release state upon an ex vivo exposure to a stimulus. The final dosage form further includes a chamber at least substantially within the outer layer and configured to carry the medicament. The final dosage form includes the medicament. The final dosage form may include an indicator element configured to indicate an exposure of the release element to the stimulus.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of U.S. patent application Ser. No. To be assigned, titled INDIVIDUALIZABLE DOSAGE FORM, naming Mahalaxmi Gita Bangera, Edward S. Boyden, Roderick A. Hyde, Muriel Y. Ishikawa, Dennis J. Rivet, Elizabeth A. Sweeney, Lowell L. Wood, Jr., and Victoria Y. H. Wood as inventors, filed Sep. 16, 2008, which is currently co-pending, or is an application of which a currently co-pending application is entitled to the benefit of the filing date.

For purposes of the USPTO extra-statutory requirements, the present application constitutes a continuation-in-part of U.S. patent application Ser. No. To be assigned, titled PERSONALIZABLE DOSAGE FORM, naming Mahalaxmi Gita Bangera, Edward S. Boyden, Roderick A. Hyde, Muriel Y. Ishikawa, Dennis J. Rivet, Elizabeth A. Sweeney, Lowell L. Wood, Jr., and Victoria Y. H. Wood as inventors, filed Sep. 16, 2008, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### SUMMARY

An embodiment of the subject matter described herein provides a final dosage form for delivering a medicament to an animal. The final dosage form includes an outer layer. The final dosage form includes a release element configured in a first medicament-release state and modifiable to a second medicament-release state upon an ex vivo exposure to a stimulus. The final dosage form includes a chamber at least substantially within the outer layer and configured to carry the medicament. The final dosage form includes the medicament. The final dosage form may include an indicator element configured to indicate an exposure of the release element to the stimulus.

In an embodiment, a final dosage form for delivering a medicament to an animal is described herein. The final dosage form includes an outer layer. The final dosage form includes a release element configured in a first medicament-release state and modifiable to a second medicament-release state upon an ex vivo exposure to a stimulus. The final dosage form includes a chamber at least substantially within the outer layer and configured to carry the medicament. The final dosage form includes the medicament. The final dosage form includes a containment element configured to retain the medicament within the final dosage form at least until the dosage form is administered to the animal. The final dosage form may include an indicator element configured to indicate an exposure of the release element to the stimulus.

In an embodiment, an article of manufacture is described herein that includes a package containing a final dosage form. The final dosage form includes a medicament, an outer layer, and a chamber at least substantially within the outer layer and configured to carry the medicament. The final dosage form includes a release element configured in a first medicament-release state and modifiable to a second medicament-release state upon an ex vivo exposure to a stimulus. The article of manufacture includes an instruction for preparation of the final dosage form for an efficacious administration to an animal by an ex vivo exposure of the release element of the final dosage form to the stimulus. The final dosage form may include a containment element configured to retain the medicament within the final dosage form at least until the final dosage form is introduced into the animal. The final dosage form may include an indicator element configured to indicate an exposure of the release element to the stimulus.

An embodiment includes a method of modulating a medicament-release characteristic of a final dosage form. The method includes providing an ex vivo stimulus to a release element of the final dosage form. The final dosage form includes a medicament, and an outer layer. The final dosage form also includes a release element configured in a first medicament-release state and modifiable to a second medicament-release state upon an ex vivo exposure to a stimulus. The final dosage form further includes a chamber defined at least substantially within the outer layer and configured to carry the medicament. The final dosage form may include a containment element configured to retain the medicament within the final dosage form at least until administration of the final dosage form into the animal.

An embodiment includes a method of fulfilling a request specifying a dose of a medicament for an individual animal. The method includes choosing, pursuant to the request, an instance of a final dosage form that includes the medicament. The method includes selecting a stimulus effective to change a medicament-release state of a release element of the final dosage form. The method includes initiating an ex vivo exposure of the release element of the chosen instance of the final dosage form to the selected stimulus. The final dosage form includes an outer layer, the medicament, and a chamber defined at least substantially within the outer layer and configured to carry the medicament. The final dosage form includes the release element configured in a first medicament-release state and changeable to a second medicament-release state upon an ex vivo exposure to a stimulus. The method may include verifying the ex vivo exposure of the release element of the chosen instance of the final dosage form to the selected stimulus. The method may include dispensing the chosen instance of the final dosage form after the ex vivo exposure of the release element of the chosen instance of the final dosage form to the selected stimulus as described above. The final dosage form may include a containment element configured to retain the medicament within the final dosage form at least until administration of the final dosage form into the animal.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 illustrates an example environment that includes an animal, a cross-sectional view of an example final dosage form for delivering a medicament to the animal, and an example stimulation source operable to emit a stimulus;

FIG. 2 illustrates another example environment that includes the animal, a cross-sectional view of an example final dosage form for delivering a medicament to the animal, and the example stimulation source operable to emit the stimulus;

FIG. 3 illustrates a further example environment that includes the animal, a cross-sectional view of an example final dosage form for delivering a medicament to the animal, and the example stimulation source operable to emit the stimulus;

FIG. 4 illustrates another example environment that includes the animal, a cross-sectional view of an example final dosage form for delivering a medicament to the animal, and the example stimulation source operable to emit the stimulus;

FIG. 5 illustrates a further example environment that includes an animal, a cross-sectional view of an example final dosage form for transporting medicament to the animal;

FIG. 6 illustrates an example environment that includes an article of manufacture;

FIG. 7 illustrates an example operational flow modulating a medicament-release characteristic of a final dosage form;

FIG. 8 illustrates an alternative embodiment of the operational flow of FIG. 7;

FIG. 9 illustrates an example operational flow fulfilling a request specifying a dose of a medicament for an individual animal;

FIG. 10 illustrates an alternative embodiment of the example operational flow of FIG. 9;

FIG. 11 illustrates another alternative embodiment of the example operational flow of FIG. 9

FIG. 12 illustrates a further embodiment of the example operation of FIG. 9;

FIG. 13 illustrates another embodiment of the example operational flow of FIG. 9; and

FIG. 14 illustrates a further embodiment of the example operational flow of FIG. 9.

### DETAILED DESCRIPTION

In the following detailed description, reference is made to the accompanying drawings, which form a part hereof. In the drawings, similar symbols typically identify similar components, unless context dictates otherwise. The illustrated embodiments described in the detailed description, drawings, and claims are not meant to be limiting. Other embodiments may be utilized, and other changes may be made, without departing from the spirit or scope of the subject matter presented here.

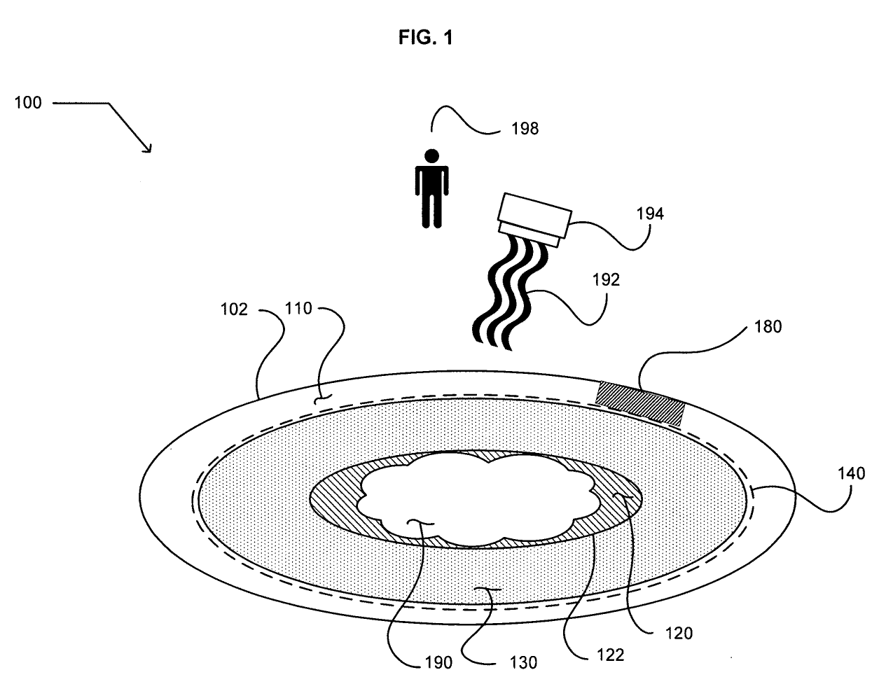

FIG. 1 illustrates an environment 100 that includes an animal 198, a cross-sectional view of an example final dosage form 102 for delivering a medicament 190 to the animal, and an example stimulus source 194 configured to emit a stimulus 192. In an embodiment, the final dosage form includes a dosage form having completed a manufacturing or production process. In an embodiment, the final dosage form includes a product, finished tablet, or capsule ready for distribution to a hospital, pharmacy, or retail store for individualizing to a particular animal, such as the animal 198. In an embodiment, the final dosage form may include a tablet shape, a spherical shape, or an ellipsoidal shape. In an embodiment, the final dosage form may include a structure or a particle carryable or transportable by a liquid or other fluid carrier.

The final dosage form 102 includes an outer layer 110, a release element 130, and a chamber 120. The release element 130 is configured in a first medicament-release state and modifiable to a second medicament-release state upon an ex vivo exposure to a stimulus. For example, the stimulus may include the stimulus 192. The chamber 120 includes a chamber wall 122, which is at least substantially within the outer layer 110, and is configured to carry the medicament 190. In an embodiment, the final dosage form 102 includes an intermediate outer layer (not shown) with the release element interposed between the outer layer and the intermediate outer layer, and the chamber is at least substantially within the intermediate outer layer (not shown).

In an embodiment, the outer layer 110 of the final dosage form 102 includes an outer layer of at least one of a tablet, capsule, particle, or solid final dosage form. In an embodiment, the outer layer 110 includes an outer peripheral layer. FIG. 1 illustrates an example embodiment where the outer layer 110 includes an outer layer around the chamber wall 122 and the release element 130. In an embodiment, the outer layer 110 is configured for administration to the animal 198 by at least one of an oral, enteral, inhalation, or implant route. In an embodiment, an enteral route includes a rectal route, such as by a rectal suppository. In an embodiment, the outer layer 110 includes an outer layer configured to release the medicament in an in vivo environment of the animal. In an embodiment, the outer layer 110 includes an outer surface. In an embodiment, the outer layer includes an outer surface of a biocompatible medicament delivery vehicle or transport.

In an embodiment, the outer layer 110 of the final dosage form 102 includes an erodible outer layer. Formulations of erodible dosage forms are known in the art. In an embodiment, the erodible outer layer includes an erodible outer layer that is at least one of soluble, permeable, or disintegrable within the animal 198. In an embodiment, the erodible outer layer includes an erodible outer layer having at least a portion that is at least one of soluble, permeable, or disintegrable in response to an acidic environment within the animal. In an embodiment, the erodible outer layer includes an erodible outer layer having at least a portion that is at least one of soluble, permeable, or disintegrable in response to a basic environment within the animal.

In an embodiment, the outer layer 110 of the final dosage form 102 includes an outer portion of a particle. Examples of such a particle include hydrogels, microspheres, polymeric microspheres, and nanoparticles as described in Lin et al., Hydrogels in controlled release formulations. Network design and mathematical modeling, Advanced Drug Delivery Reviews 58 (2006) (1379-1408). In an embodiment, the outer layer 110 of the final dosage form 102 includes an outer portion of a molecule. An embodiment includes an outer layer 110 configured to allow an in vivo discharge of at least a portion of the medicament 190 from the chamber 120 after an exposure of the release element 130 to the stimulus 192. An embodiment includes an outer layer 110 configured in cooperation with the release element 130 to allow an in vivo discharge of at least a portion of the medicament 190 from the chamber 120 after an exposure of the release element 130 to the stimulus. In an embodiment, the outer layer 110 includes an outer layer of at least a portion of the release element. In an embodiment, the release element forms the outer layer. An embodiment includes an outer layer configured to contain the medicament until the final dosage form is administered into the animal.

In an embodiment of the release element 130, the first medicament-release state is configured to retard medicament release in vivo and the second medicament-release state is configured to allow medicament release in vivo. In an embodiment of the release element 130, the first medicament-release state is configured to allow medicament release in vivo and the second medicament-release state is configured to retard medicament release in vivo.

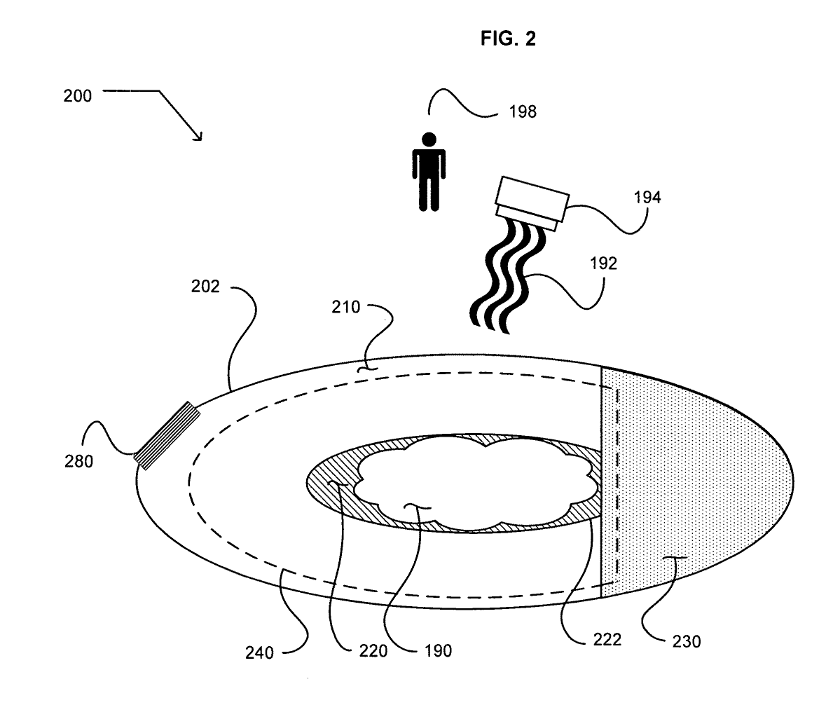

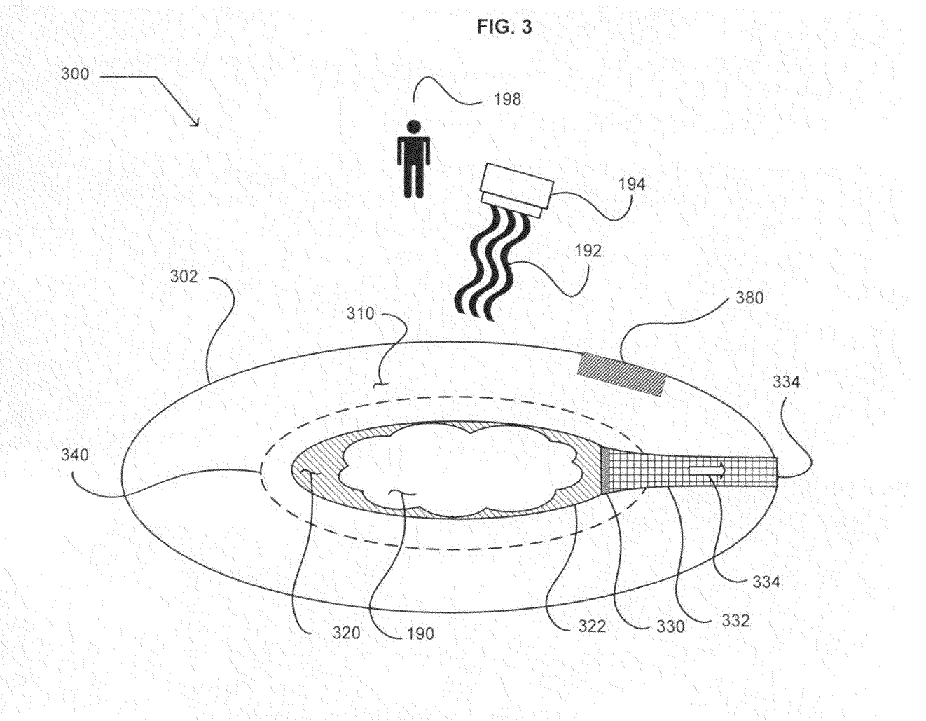

FIG. 1 illustrates a release element 130 disposed within the outer layer 110. In an embodiment, the release element includes a release element that is at least partially disposed within the outer layer, or a release element that is not disposed within the outer layer. For example, FIG. 2 infra, illustrates an example of a final dosage form 202 that includes a release element 230 that is not disposed within the outer layer 210. FIG. 3, infra, illustrates an example of a final dosage form 302 that includes a release element 330 disposed at least partially within the outer layer 310.

Returning to FIG. 1, in an embodiment, a release element 130 may be configured in a first medicament-release state and modifiable to a second medicament-release state upon an ex vivo exposure to a stimulus. An embodiment includes a release element configured in a first medicament-release state and reconfigurable to a second medicament-release state upon an ex vivo exposure to a stimulus.

In an embodiment, the release element 130 includes a release element configured in a first medicament-release state and modifiable to a second medicament-release state upon an ex vivo exposure to a non-ionizing radiation, illustrated as the stimulus 192. In an embodiment, the release element 130 is configured in a first medicament-release state and modifiable to a second medicament-release state upon an ex vivo exposure to an electromagnetic radiation, illustrated as the stimulus 192. In an embodiment, the release element 130 is configured in a first medicament-release state and modifiable to a second medicament-release state upon an ex vivo exposure to a light radiation, also illustrated as the stimulus 192. For example, light radiation may include at least one of the spectrum of ultraviolet (UV), visible light, and/or infrared (IR). In an embodiment, the release element 130 includes, but is not limited to, at least one of a poly(vinyl alcohol), gel, gel matrix, hydrogel, or azopolymer membrane. Examples of poly(vinyl alcohol) are described in (S. P. Vijayalakshmi, et al., Photodegradation of poly(vinyl alcohol) under UV and pulsed-laser irradiation in aqueous solution, JOURNAL OF APPLIED POLYMER SCIENCE, Vol. 102, No. 2, 958-966, 2006). Examples of photoresponsive polymers are described in (J. Kyoo Lee, et. al., Photo-Triggering of the Membrane Gates in Photo-Responsive Polymer for Drug Release, ENGINEERING IN MEDICINE AND BIOLOGY SOCIETY, (27th Annual International Conference) 2005 Pages: 5069-5072 (2005). In an embodiment, the release element includes a photo-labile bond between a molecule of the medicament 190 and a bioactivity inhibiting molecule that is configured in a first medicament-release state and modifiable to a second medicament-release state upon an ex vivo exposure of the labile bond to a stimulus. Examples of such a photo-labile bond are described in M. Scwarcznski, et al., Development of first photo responsive prodrug of paclitaxel, 16 BIOORGANIC & MEDICAL CHEMISTRY LETTERS, Issue 17 4492-4496 (September 2006): Epub 27 Jun. 2006. In addition, the release element may include at least one of an additional appropriate photodegradable and/or biocompatible barrier forming material.

In an embodiment, the release element 130 includes a release element configured in a first medicament-release state and modifiable to a second medicament-release state upon an ex vivo exposure to an energetic stimulus, also illustrated as stimulus 192. In an embodiment, an energetic stimulus may include at least one of a mechanical stimulus, a non-ionizing radiation stimulus, an ionizing radiation stimulus, a chemical stimulus, an acoustic stimulus, an ultrasound stimulus, a radio wave stimulus, a microwave stimulus, a light wave stimulus, or a thermal stimulus.

In an embodiment, the release element 130 is configured in a first medicament-release state and modifiable to a second medicament-release state upon an ex vivo exposure to at least one of terahertz radiation, microwave radiation, and radio wave radiation, also illustrated as the stimulus 192. For example, radio wave radiation may include, for example, at least one of ultra-high frequency radio waves (UHF), very high frequency radio waves (VHF), radio frequency (RF), and/or extremely low frequency (ELF) radio waves. In an embodiment, the release element 130 includes at least one of a foil, gold foil, a liposome, wax, dielectric/wax composite. An example of a microwave responsive liposome is described in U.S. Pat. No. 4,801,459 to R. Liburdy. An example of a microwave responsive material, including a wax and a wax/dielectric composite, is described in United States Patent Application Publication No. 2005/0191708 to R. Saul, et al. In an embodiment, the release element is configured in a first medicament-release state and modifiable to a second medicament-release state upon an ex vivo exposure to a magnetic stimulus. In an embodiment, the release element is configured in a first medicament-release state and modifiable to a second medicament-release state upon an ex vivo exposure to an electric field stimulus.

In an embodiment, the release element is configured in a first medicament-release state and modifiable to a second medicament-release state upon an ex vivo exposure to a chemical stimulus (not shown). For example, a chemical stimulus may include at least one of a stimulus based on pH change, enzymatic exposure or catalysis. In an embodiment, a chemical stimulus may include a stimulus operable to release or reverse a cooperative or a reversible molecular binding, or a stimulus operable to form an irreversible binding.

In an embodiment, the release element 130 is configured in a first medicament-release state and modifiable to a second medicament-release state upon an ex vivo exposure to a mechanical agitation stimulus (not shown). For example, a mechanical agitation stimulus may include a shaking or spinning to rupture a membrane or foil. In an embodiment, a release element is configured in a first medicament-release state and modifiable to a second medicament-release state upon an ex vivo exposure to a mechanical stimulus (not shown). For example, a mechanical stimulus may include shaking a piercing member against a foil release element. In an embodiment, the release element is configured in a first medicament-release state and modifiable to a second medicament-release state upon an ex vivo exposure to a stimulus, the release element including a mechanically activatable structure (not shown). For example, the mechanically activatable structure may include a foil or a pressure-rupturable membrane, or a heat-activatable structure.

In an embodiment, the release element 130 is permeated, dissolved, or disintegrated in response to the stimulus. In an embodiment, a release element is changed such that it is permeated, dissolved, or disintegrated in response to an in vivo environment of the animal 198 where it would not have been so before exposure to the stimulus. In an embodiment, a release element is changed such that it forms a barrier, or is impermeable, solid, or integral in response to the exposure to the stimulus where it would not have been so before the exposure to the stimulus.

In an embodiment, the release element 130 is configured in a first medicament-release state and modifiable to a second medicament-release state upon an ex vivo exposure to at least one of a thermal, acoustic stimulus and ultrasound. Examples of an acoustically active release element formed by conjugating liposomes and microbubbles are described in A. Kheirolomoom, et al., Acoustically-active microbubbles conjugated to liposomes: Characterization of a proposed drug delivery vehicle, 118 J CONTROL RELEASE, Issue 3, April 23; 118(3):275-284. Epub 2006 Dec. 23.

In an embodiment, the release element 130 includes a release element configured in a first medicament-release state and modifiable to a second medicament-release state upon an ex vivo exposure to at least one of an activation stimulus, or an actuation stimulus. In an embodiment, the release element is configured in a first medicament-release state and modifiable to a second medicament-release state upon an ex vivo exposure to a de-activation stimulus.

In an embodiment, the release element 130 includes a release element configured in a first medicament-release state and modifiable to a second medicament-release state upon an ex vivo exposure to an ultrasound stimulus. For example, the release element may include at least one of liposomes, lipid microspheres, microbubbles, lipospheres, or liposomes responsive to an ultrasound stimulus, which are described in U.S. Pat. No. 6,416,740 to Unger. In an embodiment, the release element includes at least one of polyanhidrides, polyglycolides, polyactides, poly(vinyl acetate), poly(glycolic acid), poly(ethylene), poly(lactic acid), or chitosan. An example of ultrasound-responsive polymer is described in J. Kost, et al., Ultrasound-enhanced polymer degradation and release of incorporated substances, 86 PROCEEDINGS OF THE NATIONAL ACADEMY OF SCIENCES OF THE USA, 7663-7666 (1989). An example of ultrasound-responsive polymer is described in J. Kost, et al., Ultrasonically controlled polymeric drug delivery, Makromolekulare Chemie 19 MACROMOLECULAR SYMPOSIA 275-285 (1988). An example of ultrasound-responsive chitosan is described in M. Tsaih, et al., Effect of the degree of deacetylation of chitosan on the kinetics of ultrasonic degradation of chitosan; 90 JOURNAL OF APPLIED POLYMER SCIENCE 3526-3531 (2003).

In an embodiment, the release element 130 includes at least one of polymeric micelle, liposomes, lipid microsomes, polymeric microsphere, nanoparticles, cyclodextrin, gel, gel matrix, hydrogel, or cellulose. Examples of polymeric micelles are described in U.S. Pat. No. 7,229,973 to Bae, et al. Examples of polymer microspheres are described in U.S. Pat. No. 5,718,921 to Mathiowitz, et al. Examples of cyclodextrin are described in U.S. Pat. No. 7,270,808 to Cheng, et al., titled “Cyclodextrin-based polymers for therapeutics delivery.” Examples of hydrogels are described in Lin et al., Hydrogels in controlled release formulations: Network design and mathematical modeling, ADVANCED DRUG DELIVERY REVIEWS 58 (2006) 1379-1408). Examples of cellulose are described in U.S. Pat. No. 6,821,531 to Kumar.

In an embodiment, the release element 130 includes a release element enclosing the chamber 120, configured in a first medicament-release state, and modifiable to a second medicament-release state upon an ex vivo exposure to a stimulus, illustrated as the stimulus 192. For example, FIG. 1 illustrates an embodiment where the outer layer 110 has a spherical shape, the chamber may have similar nested spherical shape, and the release element having a spherical shape and surrounding the chamber. However, nothing in this document expresses or implies a required similarity of shape among one or more of the chamber, the release element, or the outer layer. For example, an embodiment may include a liposome forming the release element and functionally defining a chamber.

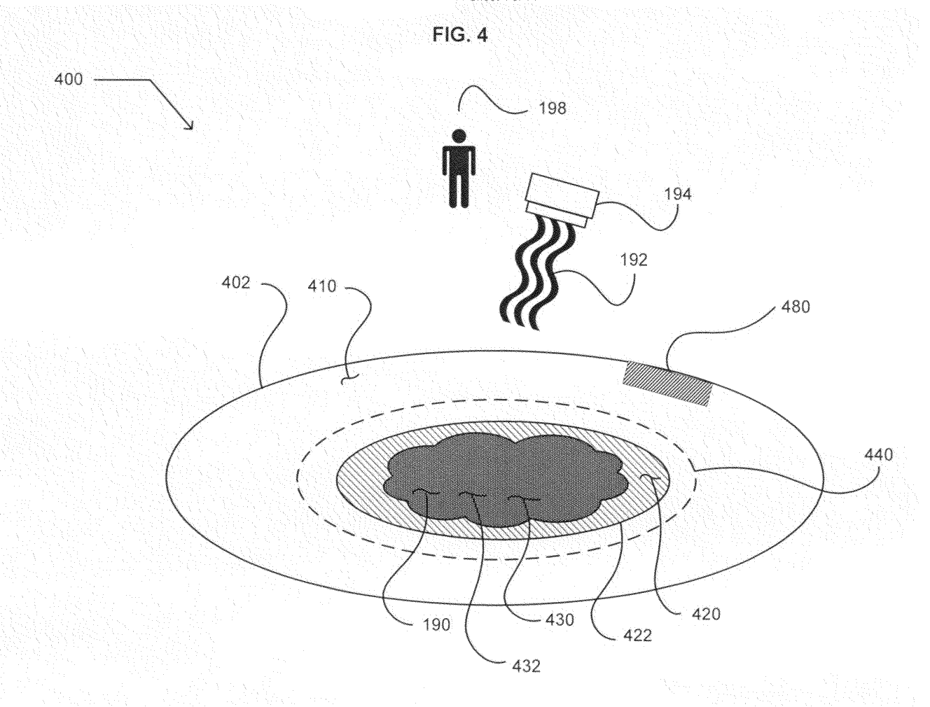

In an embodiment, the release element 130 includes a release element encapsulating the chamber. In an embodiment, the release element includes a release element encapsulating the medicament 190 in cooperation with the chamber wall 122, configured in a first medicament-release state, and modifiable to a second medicament-release state upon an ex vivo exposure to a stimulus. For example, FIG. 2, infra, illustrates a release element 230 encapsulating a medicament 190 in cooperation with a chamber 220 as expressed by a chamber wall 222. In an embodiment, the release element includes a release element obstructing an aperture of the chamber. For example, FIG. 3, infra, illustrates a release element 330 in cooperation with a chamber 320 as expressed by a chamber wall 322 obstructing an aperture 332 of the chamber and preventing a discharge of a medicament 190 along a fluid communication path 336 In an embodiment, the release element includes at least two particles each collectively or respectively forming a chamber carrying a respective instance of the medicament. For example, FIG. 4, infra, illustrates a release element 430 that includes at least two particles 432 each collectively or respectively forming a chamber carrying an instance of the medicament 190. The at least two particles are configured in a first medicament-release state, and modifiable to a second medicament-release state upon an ex vivo exposure of the at least two particles to a stimulus. For example, the at least two particles may include at least one of hydrogels, liposomes, or dendrimers configured to carry the medicament in an association with their pores, interstitial cavities, structural interstices, bonds, or amorphous cavities.

In an embodiment, the release element includes a labile bond between a molecule of the medicament and a bioactivity inhibiting molecule configured in a first medicament-release state and modifiable to a second medicament-release state upon an ex vivo exposure of the labile bond to a stimulus (not shown).

Referring again to FIG. 1, in an embodiment, the release element 130 is configured in a first medicament-release state and modifiable to a second medicament-release state upon an ex vivo exposure to a stimulus, and configured to contain the medicament 190 at least until the final dosage form 102 is administered into the animal 198.

FIG. 1 illustrates an embodiment having the chamber 120 formed within the outer layer 110 and configured to carry the medicament 190. In an embodiment, the chamber is at least substantially defined within the outer layer and configured to carry the medicament until released by the release element. For example, FIG. 2 illustrates an embodiment that includes the chamber 220 at least substantially defined within the outer layer 210 and configured to carry the medicament 190 until released by the release element 230. FIG. 3 illustrates an embodiment that includes the chamber 320 at least substantially defined within the outer layer 310 and configured to carry the medicament 190 until released by the release element 330.

In an embodiment (not shown), the release element and chamber both may be formed by a particle, such as a liposome, or a hydrogel. In such embodiment, the chamber includes at least one chamber at least substantially within the outer layer of the particle and configured to carry the medicament. In an embodiment (not shown), the chamber includes at least two chambers at least substantially within a particle and configured to carry respective instances of the medicament.

In an embodiment (not shown), the chamber 120 includes a first chamber configured to carry a first medicament and a second chamber configured to carry a second medicament. In an embodiment, the chamber includes a chamber configured to confine the medicament in cooperation with the release element. In an embodiment (not shown), the chamber includes at least one chamber configured to confine the medicament in a structural cooperation with the release element. In an embodiment (not shown), the chamber is configured to initially carry the medicament. The chamber is also configured to release at least a portion of the medicament upon at least one of a reconfiguration, bursting, puncture, permeation, dissolution, and disintegration of the release element 130. In an embodiment (not shown), the chamber includes a first chamber configured to carry a first constituent of the medicament and a second chamber configured to carry a second constituent of the medicament. In an embodiment (not shown), the chamber includes a first chamber configured to carry a first reactant of the medicament and a second chamber configured to carry a second reactant of the medicament. In an embodiment (not shown), a combination of the first reactant and the second reactant in response to an ex vivo exposure of the release element initiates a chemical activation of the medicament and a physical releasability of the medicament. In an embodiment (not shown), a combination of the first reactant and the second reactant in response to an ex vivo exposure of the release element initiates a chemical activation of the medicament but does not provide a physical releasability of the medicament. The physical releasability of the medicament by another ex vivo exposure of the dosage form to a stimulus.

In an embodiment shown in FIG. 1, the final dosage form 102 includes a containment element 140 configured to retain the medicament 190 within the final dosage form until the dosage form is administered to the animal 198. In embodiment, the containment element may include a separate structure, such as a film or coating, configured to retain the medicament. Such a containment element 140 may form an exterior layer over the outer layer 110, or may form a layer interposed between the outer layer 110 and the chamber 120. In an embodiment, the containment element 140 may inhibit a discharge of the medicament 190 from the final dosage form 102 prior to its introduction into the animal 198, without regard to whether the release element is in its first medicament-release state or its second medicament-release state. In an embodiment, the containment element 140 includes a containment element 140 configured to retain the medicament 190 within the final dosage form 102 until the final dosage form 102 is exposed to an in vivo environment in the animal 198, and to modulate a release of at least a portion of the medicament 190 in vivo upon delivery of the final dosage form 102 to the animal 198.

In an embodiment, the containment element may be formed by a combination of the outer layer 110 and the release element 130.

In an embodiment, the containment element 140 includes a containment layer configured to encapsulate the medicament 190 within the final dosage form 102 until the final dosage form is administered to the animal 198. For example, the containment element 140 may include a coating covering the outer layer 110 of the final dosage form 102, such as an enteric coating configured to prevent a release of the medicament from the final dosage form until the final dosage form is administered to the animal. In another example, the containment element 140 may include a coating covering the release element 130 of the final dosage form 102. In an embodiment, the containment element includes a containment envelope configured to retain the medicament within the final dosage form until the dosage form is administered to the animal.

In an embodiment illustrated in FIG. 2 infra, the containment element 240 includes a containment element 240 configured to prevent a release of the medicament 190 from the final dosage form 202 until the final dosage form 202 is introduced into the animal 198.

Returning to FIG. 1, in an embodiment, the medicament 190 includes at least one of an agent, treatment agent, drug, prodrug, therapeutic, nutraceutical, medication, vitamin, nutritional supplement, medicine, remedy, medicinal substance, or cosmetic. In an embodiment, the medicament includes a first component of the medicament and a second component of the medicament. In an embodiment, the medicament includes a first reactant of the medicament and a second reactant of the medicament. In an embodiment, the medicament includes at least one prodrug and optionally an activating-enzyme of the prodrug. In an embodiment, the chamber includes a first chamber configured to carry a prodrug, and a second chamber configured to carry an activating enzyme of the prodrug.

In an embodiment, the final dosage form 102 may further include an indicator element 180 configured to indicate an exposure of the release element 130 to the stimulus 192. In an embodiment, the indicator element 180 includes an indicator element 180 configured to optically indicate an exposure of the release element to the stimulus 192 by at least one of dielectric, a conductivity, or ultrasonic profile responsive to an exposure of the release element to the stimulus. The indicator element 180 including, for example, at least one of 4-keto-bacteriorhodopsin films, cinnamylidene acetyl chloride, a-methylcinnamylidene acetyl chloride, α,γ-dimethylcinnamylidene acetyl chloride, a-phenylcinnamylidene acetyl chloride, a-phenoxycinnamylidene acetyl chloride, and cyanocinnamylidene acetyl chloride, leuco dye-serum albumin albumin complexes, azo dyes, or poly(ethylene glycol). Examples of bacteriorhodopsin films are described in A. Druzhko, et al., 4-Keto-bacteriorhodopsin films as a promising photochromic and electrochromic biological material, BIOSYSTEMS. 1995; 35(2-3): 129-32. Examples of hydrophilic photosensitive polymers are described in U.S. Pat. No. 5,990,193 to Russell, et al. Examples of photosensitive compositions for detection of radiation in the ultraviolet wavelength, including leuco dye-serum albumin complexes, are described in U.S. Pat. No. 4,466,941 to Cerami, et al. Examples of using azo dye for an indicator is described in U.S. Pat. No. 5,679,442. Examples of poly(ethylene glycol) are described in U.S. Pat. No. 5,990,193 to Russell, et al., and in Zhong, et al., Photodegradation Behavior of Polycaprolactone-Poly(ethylene glycol) Block Copolymer, Vol. 10, No. 4 CHINESE CHEMICAL LETTERS 327-330 (1999).

In an embodiment depicted in FIG. 1, the indicator element 180 includes an electronically-detectable indicator element 180 configured to indicate an exposure of the release element 130 to the stimulus 192. For example, the electronically-detectable indicator element 180 may include a substance, material, or device having a conductive property that makes an electronically-detectable change in response to an exposure to the stimulus 192. An example of such substance, material, or device includes a shape memory alloy switch that responds to heat described in U.S. Pat. No. 5,410,290 to Cho. Other examples of such substances, materials, or devices include a material that polymerizes in the presence of an ultrasound and changes a conductive property in response, such as the ultrasonic polymerization of methyl methacrylate described in U.S. Pat. No. 5,466,722 to Stoffer, et al., the heat or UV radiation triggered polymerization of acrylamide, or the microwave triggered polymerization of trimethylene carbonate. Another example of such substances, materials, or devices include the use of bistable compounds whose conductivity changes based upon exposure to electromagnetic radiation as described in U.S. Pat. No. 7,175,961 to Beck, et al. Another example includes a metal film or foil degradable by microwaves to release the medication whose state can degradation detected electrically.

In an embodiment, the indicator element 180 includes an electronically-detectable indicator element 180 configured to indicate an exposure of the release element 130 to the stimulus 192. For example, the electronically-detectable indicator element 180 may include a dielectric element having a property that makes an electronically-detectable change in response to an exposure to the stimulus 192. An example of such a dielectric element may include a one-time programmable memory cell described in U.S. Pat. No. 7,256,446, to Hu, et al., or a switch comprising microelectromechanical elements described in U.S. Pat. No. 7,336,474 to Lerche, et al.

In an example, the electronically-detectable indicator element 180 may include an element having a permittivity that makes an electronically-detectable change in response to an exposure of the release element to the stimulus 192. An example of such an element having a permittivity may include photonic crystals whose permittivity changes through the addition of photonic and/or electrical energy as described in U.S. Pat. No. 6,859,304 to Miller, et al.

In another example, the electronically-detectable indicator element 180 may include an element having an ultrasonic profile that makes an ultrasound-discernable change in response to an exposure of the release element to the stimulus 192. An example of an element having an ultrasonic profile that includes a polymer monitorable using the continuous wave ultrasonic process monitor is described in U.S. Pat. No. 7,017,412 to Thomas, et al. Another example of an element having an ultrasonic profile that includes a polymer monitorable using the apparatus for degree on doneness is described in U.S. Pat. No. 7,191,698 to Bond, et al. A further example of an element having an ultrasonic profile that includes a degradable metal film or metal foil.

In another example, the electronically-detectable indicator element 180 may include a carrier, admixture, or excipient having a property that makes an ultrasound-discernable change in response to an exposure of the release element to the stimulus 192. For example, an admixture may include a phase change material (PCM) as an inert filler and having a property that makes an ultrasound-discernable change in response to an exposure of the release element to ultrasound. Examples of such PCMs include polyvinyl alcohol (PVA)-stearic acid (SA) and polyvinyl chloride (PVC)-stearic acid (SA). An example of Polymer-stearic acid blend is described in Ahmet Sari, et al., Polymer-stearic acid blends as form-stable phase change material for thermal energy storage, 64 JOURNAL OF SCIENTIFIC & INDUSTRIAL RESEARCH, at pp. 991-996 (December 2005). Other examples are described in United States Patent Application No. 2007/0249753 to Lin, et al. (polyether fatty-acid ester (polyethylene glycol or polytetramethylene glycol base polymer), and U.S. Pat. No. 5,565,132 to Salyer (Addition of microwave absorber to make PCM materials sensitive to microwaves). Ultrasonic detection or discernment of phase changes in a PCM may be implemented using techniques described by A. W. Aziz, & S. N. Lawandy, Ultrasonic detection of segmental relaxations in thermoplastic polyurethanes, 31 JOURNAL OF APPLIED POLYMER SCIENCE 1585 (Issue 6, 2003) or S. L. Morton, Ultrasonic cure monitoring of photoresist during pre-exposure bake process, ULTRASONICS SYMPOSIUM, 1997. PROCEEDINGS., 1997 IEEE Volume 1, at 837-840 (October 1997).

FIG. 2 illustrates an environment 200 that includes the animal 198, a cross-sectional view of an example final dosage form 202 for delivering the medicament 190 to the animal, and the example stimulation source 194 operable to emit the stimulus 192. In an embodiment, the final dosage form includes a dosage form having completed a manufacturing or production process. In an embodiment, the final dosage form includes a product, finished tablet, or capsule ready for distribution to a hospital, pharmacy, or retail store for individualizing to a particular animal, such as the animal 198. In an embodiment, the final dosage form may include a tablet shape, a spherical shape, or an ellipsoidal shape. In an embodiment, the final dosage form may include a structure or a particle carryable or transportable by a liquid or other fluid carrier.

The final dosage form 202 includes an outer layer 210, the release element 230, and the chamber 220 as expressed by the chamber wall 222. The release element is configured in a first medicament-release state and modifiable to a second medicament-release state upon an ex vivo exposure to a stimulus. For example, the stimulus may include the stimulus 192. The chamber includes a chamber wall 222, is at least substantially within the outer layer, and is configured to carry the medicament 190. In an embodiment, the final dosage form may include an indicator element 280. In an embodiment, the final dosage form may include a containment element 240.

The environment 200 illustrates an embodiment where the release element 230 encapsulates the medicament 190 in cooperation with the chamber 220 as expressed by the chamber wall 222. The outer layer 210 and the release-element 230 are cooperatively configured to retain the medicament 190 if the release-element is in a first medicament-release state and allow an in vivo discharge of at least a portion of the medicament from the chamber if the release-element is in a second medicament release state. In an embodiment of this example, the release element may include at least one of a poly(vinyl alcohol), gel, gel matrix, hydrogel, and azopolymer photo or light modifiable substance as described above. In an embodiment of this example, the release element may include at least one of a polyanhidride, polyglycolide, polyactide, poly(vinyl acetate), poly(glycolic acid), poly (ethylene), poly(lactic acid), chitosan, or an acoustic or ultrasound modifiable substance as described above. For example, when the first medicament-release state is configured to retard medicament release and the second medicament-release state is configured to allow medicament release in vivo, the release element when configured in the first medicament-release state will retard medicament release from the final dosage form upon delivery of the final dosage form into the animal. For example, in a first medicament release state, the release element is impermeable to the environment outside the final dosage form, and impermeable to the medicament in the chamber. Following exposure to an appropriately configured stimulus, the release element achieves a second medicament release state that is, for example, permeable to the medicament. The second medicament release state may include, for example, a state where the release element dissolves or dissipates upon exposure to an aqueous environment, gastric juices or a certain pH environment.

FIG. 3 illustrates a non-limiting environment 300 that includes the animal 198, a cross-sectional view of an example final dosage form 302 for delivering the medicament 190 to the animal, and the example stimulation source 194 operable to emit the stimulus 192. In an embodiment, the final dosage form includes a dosage form having completed a manufacturing or production process. In an embodiment, the final dosage form includes a product, finished tablet, or capsule ready for distribution to a hospital, pharmacy, or retail store for individualizing to a particular animal, such as the animal 198. In an embodiment, the final dosage form may include a tablet shape, a spherical shape, or an ellipsoidal shape. In an embodiment, the final dosage form may include a structure or a particle carryable or transportable by a liquid or other fluid carrier.

The final dosage form 302 includes an outer layer 310, a chamber 320, and a release element 330. The final dosage form also includes a release passageway 332 configured to provide a medicament communication pathway between the chamber and the environment through an aperture 334 in the outer layer. The release element is configured in a first medicament-release state and modifiable to a second medicament-release state upon an ex vivo exposure to a stimulus. For example, the stimulus may include the stimulus 192. The chamber includes a chamber wall 322, is at least substantially within the outer layer, and is configured to carry the medicament 190. In an embodiment, the final dosage form may include an indicator element 380. In an embodiment, the final dosage form may include a containment element 340.

FIG. 3 illustrates a non-limiting embodiment wherein an embodiment of the final dosage form 302 includes the release element 330 retaining the medicament 190 in cooperation with the chamber 320 as expressed by the chamber wall 322. The outer layer 310 and the release-element 330 are cooperatively configured to retain the medicament 190 if the release-element is in one medicament-release state and allow an in vivo discharge of at least a portion of the medicament from the chamber if the release-element is in another medicament release state. When the release-element is in a state the releases the medicament, the medicament may discharge or flow along the fluid communication path 336 expressed at least in part by the release passageway 332.

In an embodiment, the release element may include at least one of a poly(vinyl alcohol), gel, gel matrix, hydrogel, and azopolymer photo or light modifiable substance as described above. In an embodiment, the release element may include at least one of a foil, gold foil, wax, or dielectric/wax composite microwave modifiable substance. In an embodiment of this example, the release element may include at least one of a polyanhidride, polyglycolide, polyactide, poly(vinyl acetate), poly(glycolic acid), poly (ethylene), poly(lactic acid), chitosan, or an acoustic or ultrasound modifiable substance as described above. For example, when the first medicament-release state is configured to retard medicament release and the second medicament-release state is configured to allow medicament release in vivo, the release element when configured in the first medicament-release state will retard medicament release from the release passageway 332 and the aperture 334 of the final dosage form upon delivery of the final dosage form into the animal.

FIG. 4 illustrates an environment 400 that includes the animal 198, a cross-sectional view of an example final dosage form 402 for delivering the medicament 190 to the animal, and the example stimulation source 194 operable to emit the stimulus 192. In an embodiment, the final dosage form 402 includes a dosage form having completed a manufacturing or production process. In an embodiment, the final dosage form 402 includes a product, finished tablet, or capsule ready for distribution to a hospital, pharmacy, or retail store for individualizing to a particular animal, such as the animal 198. In an embodiment, the final dosage form 402 may include a tablet shape, a spherical shape, or an ellipsoidal shape. In an embodiment, the final dosage form 402 may include a structure or a particle carryable or transportable by a liquid or other fluid carrier.

The final dosage form 402 includes an outer layer 410, a chamber 420, and a release element 430. The release element is configured in a first medicament-release state and modifiable to a second medicament-release state upon an ex vivo exposure to a stimulus. For example, the stimulus may include the stimulus 192. The chamber includes a chamber wall 422, is at least substantially within the outer layer, and is configured to carry the medicament 190. In an embodiment, the final dosage form 402 may include an indicator element 480. In an embodiment, the final dosage form 402 may include a containment element 440.

In an embodiment, the chamber 420 includes a chamber at least substantially within the outer layer 410 and configured to carry the medicament 190. The chamber includes at least two pores, interstitial cavities, smaller chambers, interstices of a molecular structure, or amorphous cavities. In an embodiment, the chamber may include respective chambers formed by at least one of an absorbent, liposome, or hydrogel. For example, at least two particles may be located in a cavity, such as the chamber 120, and in themselves constitute a distributed chamber by an aggregation of their pores, interstitial cavities, smaller chambers, interstices of a molecular structure, or amorphous cavities. In another example, at least two microparticles may be throughout a carrier having an outer layer, each microparticle having an effective chamber. In an embodiment, the chamber is located at least substantially within the release element 430. In an embodiment, the distributed chamber is located at least substantially within the outer layer 410.

The final dosage form 402 may include a release element 430 that is proximate to the medicament 190 in the chamber 420. In an embodiment, the release element 430 may include a carrier, admixture, or excipient configured in a first medicament-release state and modifiable to a second medicament-release state upon an ex vivo exposure to a stimulus. Particles of such a carrier, admixture, or excipient may be configured to retain or bind to particles of the medicament 190 and reduce its bioavailability if the release-element 430 is in a first medicament-release state, and release from or unbind particles of the medicament 190 and allow an in vivo discharge of at least a portion of the medicament 190 from the chamber 420 if the release-element 430 is in a second medicament release state.

In an embodiment, an instance of the final dosage form 402 may carry at least two particles, small particles, or microparticles that each include a portion that forms a release element 430 modifiable by exposure to a stimulus 192, and a chamber (not shown). The chambers of the at least two particles, small particles, or microparticles each configured to carry a respective instance of the medicament, and collectively forming a distributed chamber. For example, the at least two particles, small particles, or microparticles may include hydrogels, liposomes, or dendrimers having pores, interstitial cavities, structural interstices, bonds, or amorphous cavities configurable to carry molecules of the medicament. The at least two particles, small particles, or microparticles are configured in a first medicament-release state and modifiable to a second medicament-release state upon an ex vivo exposure of the at least two particles, small particles, or microparticles to a stimulus. For example, photosensitive hydrogel particles may carry the medicament. In an embodiment, microwave sensitive liposomes may carry the medicament. In an embodiment, the release element includes a labile bond between a molecule of the medicament and molecule of a bioactivity inhibiting molecule configured in a first medicament-release state and modifiable to a second medicament-release state upon an ex vivo exposure of the labile bond to a stimulus (not illustrated).

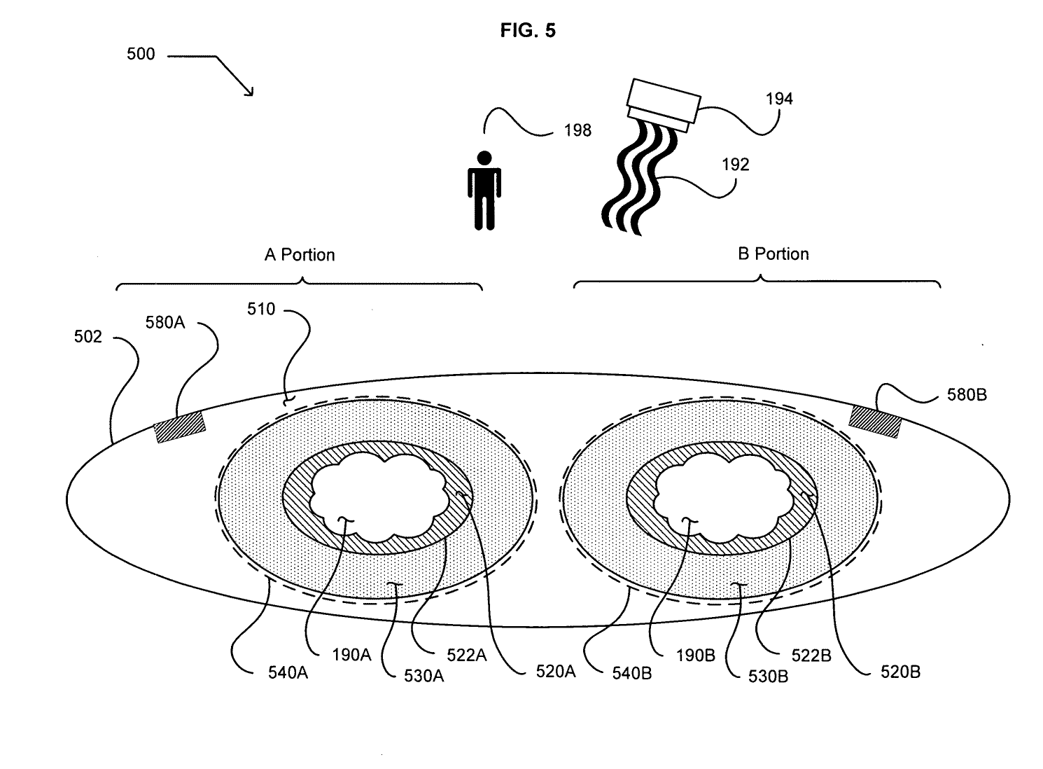

FIG. 5 illustrates an environment 500 that includes an animal 198, a cross-sectional view of a final dosage form 502 for transporting a medicament to the animal. The medicament is illustrated as a first medicament 190A and second medicament 190B. In an embodiment, the final dosage form includes a dosage form having completed a manufacturing or production process. In an embodiment, the final dosage form 502 includes a product, finished tablet, or capsule ready for distribution to a hospital, pharmacy, or retail store for individualizing to a particular animal, such as the animal 198. In an embodiment, the final dosage form 502 may include a tablet shape, a spherical shape, or an ellipsoidal shape. In an embodiment, the final dosage form 502 may include a structure or a particle carryable or transportable by a liquid or other fluid carrier.

The final dosage form 502 includes an outer layer 510, and at least two dosage elements. The at least two dosage elements are illustrated as A Portion and B Portion, and by “A” and “B” after certain reference numbers in FIG. 5. The A Portion includes a chamber 520A, a release element 530A, and a medicament 190A. In an embodiment, the A Portion includes a containment element 540A. In an embodiment, the A Portion includes an indicator element 580A. The B Portion includes a chamber 520B, a release element 530B, and a medicament 190B. In an embodiment, the B Portion includes a containment element 540B. In an embodiment, the B Portion includes an indicator element 580B.

In an embodiment, the A Portion of the final dosage form 502 may be at least substantially similar to the chamber 120, the release element 130, the containment element 140, and the indicator element 180 of FIG. 1. In an embodiment, the A Portion may be at least substantially similar to the chamber 220, the release element 230, the containment element 240, and the indicator element 280 of FIG. 2. In an embodiment, the A Portion may be at least substantially similar to the chamber 320, the release element 330, the containment element 340, and the indicator element 380 of FIG. 3. In an embodiment, the A Portion may be at least substantially similar to the chamber 420, the release element 430, the containment element 440, and the indicator element 480 of FIG. 4. Similarly, the B Portion of the final dosage form 502 may be at least substantially similar to that described in conjunction with at least one of FIG. 1, FIG. 2, FIG. 3, or FIG. 4.

In an embodiment, the first medicament 190A and the second medicament 190B may be at least substantially similar instances of one medicament. In an embodiment, the first medicament 190A and the second medicament 190B may be at least substantially similar instances of one medicament, but in at least substantially differing dosage amounts. For example, the first medicament 190A may be a 50-milligram dose of a medicament and the second medicament 190B may be a 100-milligram dose of the same medicament. In an embodiment, the first medicament 190A and the second medicament 190B may be at least substantially similar instances of one medicament, but in at least substantially differing dosage characteristics, such as a regular release formulation and a sustained release formulation. In an embodiment, the first medicament 190A and the second medicament 190B may be at least substantially different medicaments.

In use, the A Portion and the B Portion of the final dosage form 502 may be individually or collectively exposed ex vivo to a stimulus, illustrated as the stimulus 192. For example, where the first medicament 190A is a 50-milligram dose of a medicament and the second medicament 190B is a 100-milligram dose of a same medicament, where the release element 530A and release element 530B are modifiable by the same stimulus such as microwave energy, and where the first medicament-release state is configured to retard medicament release in vivo and the second medicament-release state is configured to allow medicament release in vivo, irradiation of the A Portion with microwave energy will actuate the A Portion and make 50-milligrams of the medicament available upon delivery of the final dosage form to the animal 198. Similarly, irradiation of the B Portion with microwave energy will actuate the B Portion and make 100-milligrams of the medicament available upon delivery of the final dosage form to the animal. Further, irradiation of both the A Portion and the B Portion with microwave energy will actuate both Portions and make 150-milligrams of the medicament available upon delivery of the final dosage form to the animal. In another example, the first medicament 190A is a 100-milligram dose of a first medicament and the second medicament 190B is a 100-milligram dose of a second medicament. Selective irradiation of the A Portion or the B Portion will make one or both of the medicaments bioavailable upon delivery of the final dosage form to the animal. In a further example, the release element 530A is modifiable by a first stimulus and the release element 530B is modifiable by the second and different stimulus.

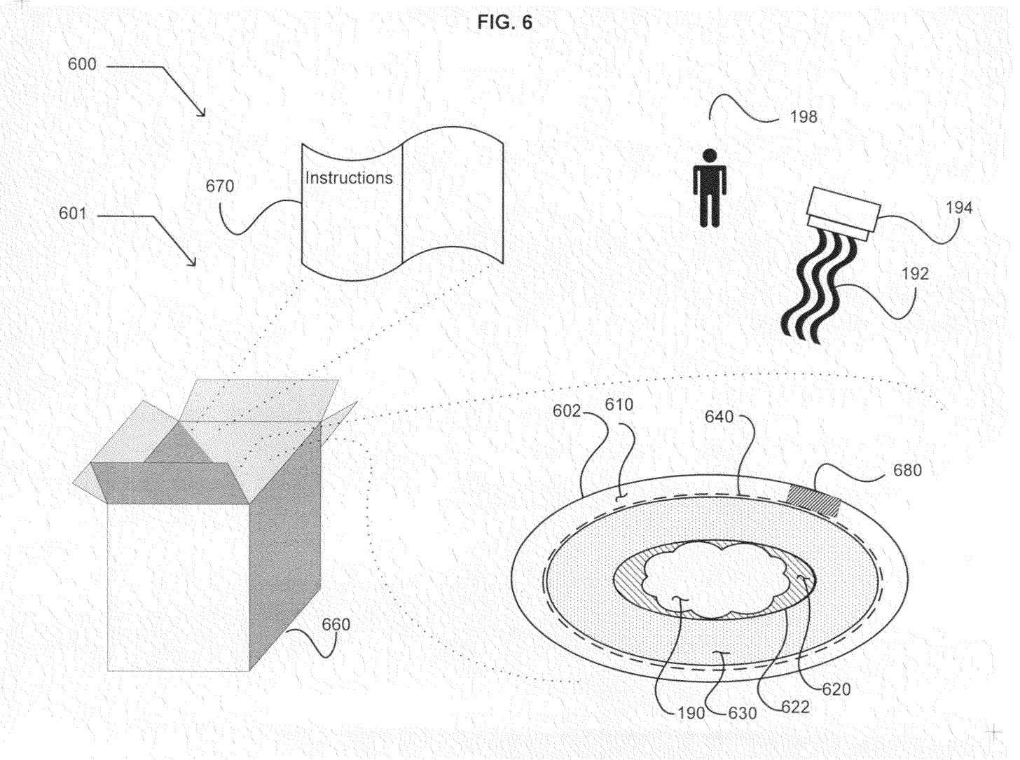

FIG. 6 illustrates an example environment 600 that includes an article of manufacture 601. The article of manufacture includes a package 660 containing a final dosage form 602 and providing an instruction 670. The final dosage form includes a medicament 190, an outer layer 610, a release element 630, and a chamber 620. The release element is configured in a first medicament-release state and modifiable to a second medicament-release state upon an ex vivo exposure to an actuation-stimulus. The chamber lies at least substantially within the outer layer and is configured to carry the medicament. The instruction includes instruction for preparation of the final dosage form for an efficacious administration to an animal by an ex vivo exposure of the release element of the final dosage form to the stimulus.

In an embodiment, the final dosage form 602 may be at least substantially similar to the final dosage form 102 of FIG. 1. In an embodiment, the final dosage form 602 may be at least substantially similar to the final dosage form 202 of FIG. 2. In an embodiment, the final dosage form 602 may be at least substantially similar to the final dosage form 302 of FIG. 3. In an embodiment, the final dosage form 602 may be at least substantially similar to the final dosage form 402 of FIG. 4. In an embodiment, the final dosage form 602 may be at least substantially similar to the final dosage form 502 of FIG. 5.

In an embodiment, the instruction 670 includes at least one of information indicating an actuation-stimulus type, an actuation-stimulus wavelength, an actuation-stimulus intensity, an actuation-stimulus duration, a spatial distribution of the stimulus relative to the final dosage form, a target-value for an exposure indicator, or a combination thereof. For example, the information indicating a spatial distribution of the stimulus relative to the final dosage form may include information corresponding to aiming the stimulus, such as toward a right hand portion, a center portion, or a left hand portion of the final dosage form. In an embodiment, the instruction includes an instruction presented by at least one of a label (not shown) on the package 660, an insert in the package, illustrated as the instruction 670, or an address to electronically published content (not shown). In an embodiment, the instruction includes instruction for preparation of the final dosage form for an efficacious administration to an animal by a human-initiated ex vivo exposure of the release element of the final dosage form to the actuation-stimulus.

In an embodiment, the final dosage form 602 further includes a containment element 640 configured to retain the medicament within the final dosage form until the final dosage form is introduced into the animal. In an embodiment, the final dosage form includes an indicator element 680 configured to indicate an exposure of the release element to the stimulus. In an embodiment, the instruction 670 includes information indicating an expected value of the indicator element.

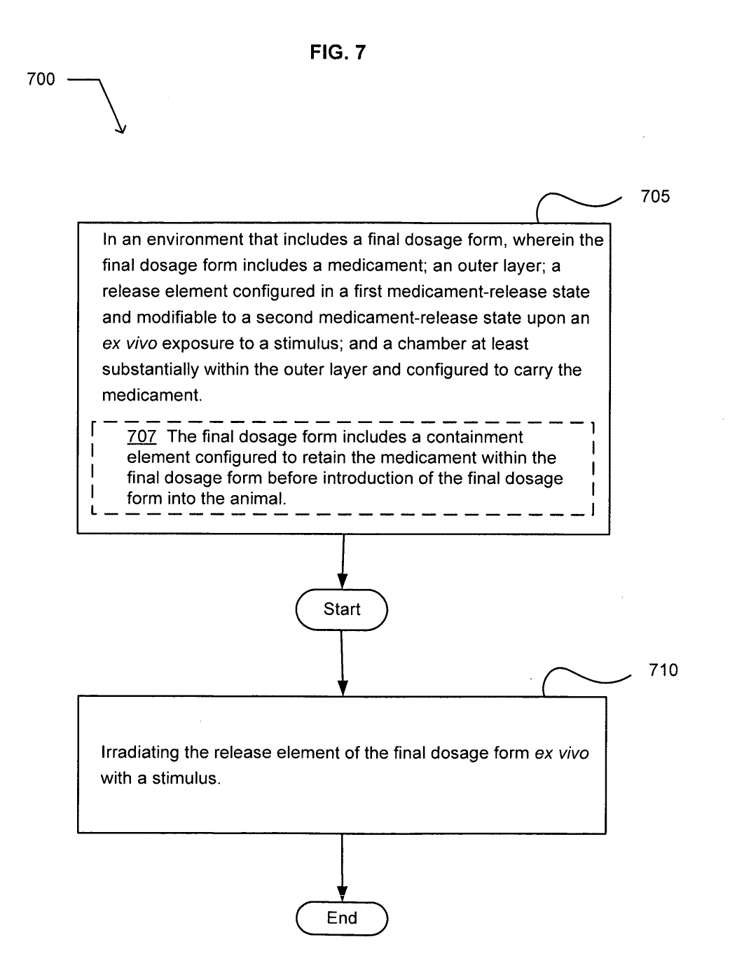

FIG. 7 illustrates an example operational flow 700 modulating a medicament-release characteristic of a final dosage form. A start operation occurs in an environment 705 that includes the final dosage form. The final dosage form includes a medicament, an outer layer, a release element configured in a first medicament-release state and modifiable to a second medicament-release state upon an ex vivo exposure to a stimulus, and a chamber at least substantially within the outer layer and configured to carry the medicament. After the start operation, the operational flow includes an individualization operation 710. The individualization operation includes irradiating the release element of the final dosage form ex vivo with a non ionizing radiation. For example, the irradiating the release element of the final dosage form ex vivo with a stimulus may occur in a hospital pharmacy, a retail pharmacy, a battlefield hospital, a veterinary facility, or other location dispensing medicaments. In another example, the irradiating a release element of the final dosage form ex vivo with a stimulus may occur in a persons home. The operational flow then proceeds to an end operation. In an alternative embodiment, the final dosage form further includes a containment element configured to retain the medicament within the final dosage form before introduction of the final dosage form into the animal.

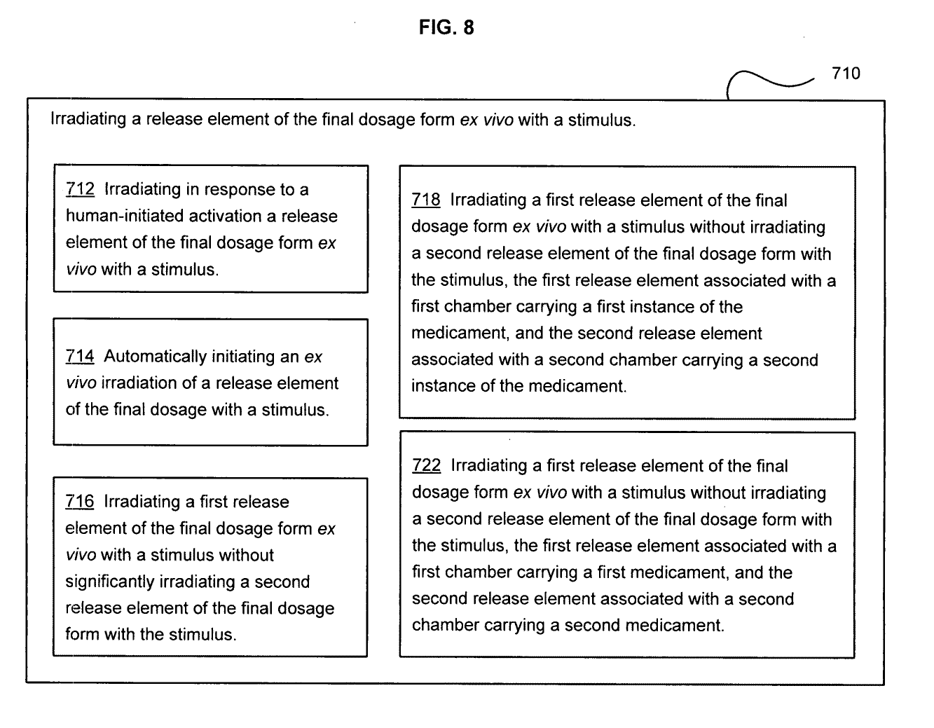

FIG. 8 illustrates an alternative embodiment of the operational flow 700 of FIG. 7. The individualization operation 710 may include at least one additional operation. The at least one additional operation may include at least one of an operation 712, an operation 714, an operation 716, an operation 718, or an operation 722. The operation 712 includes irradiating in response to a human-initiated activation a release element of the final dosage form ex vivo with a non-ionizing radiation. The operation 714 includes automatically initiating an ex vivo irradiation with a non-ionizing radiation a release element of the final dosage. The operation 716 includes irradiating a first release element of the final dosage form ex vivo with a non-ionizing radiation without irradiating a second release element of the final dosage form with the stimulus. The operation 718 includes irradiating a first release element of the final dosage form ex vivo with a non-ionizing radiation without irradiating a second release element of the final dosage form with the non-ionizing radiation. The first release element is associated with a first chamber carrying a first instance of the medicament, and the second release element is associated with a second chamber carrying a second instance of the medicament. The operation 722 includes irradiating a first release element of the final dosage form ex vivo with a non-ionizing radiation without irradiating a second release element of the final dosage form with the stimulus. The first release element is associated with a first chamber carrying a first medicament, and the second release element is associated with a second chamber carrying a second medicament.

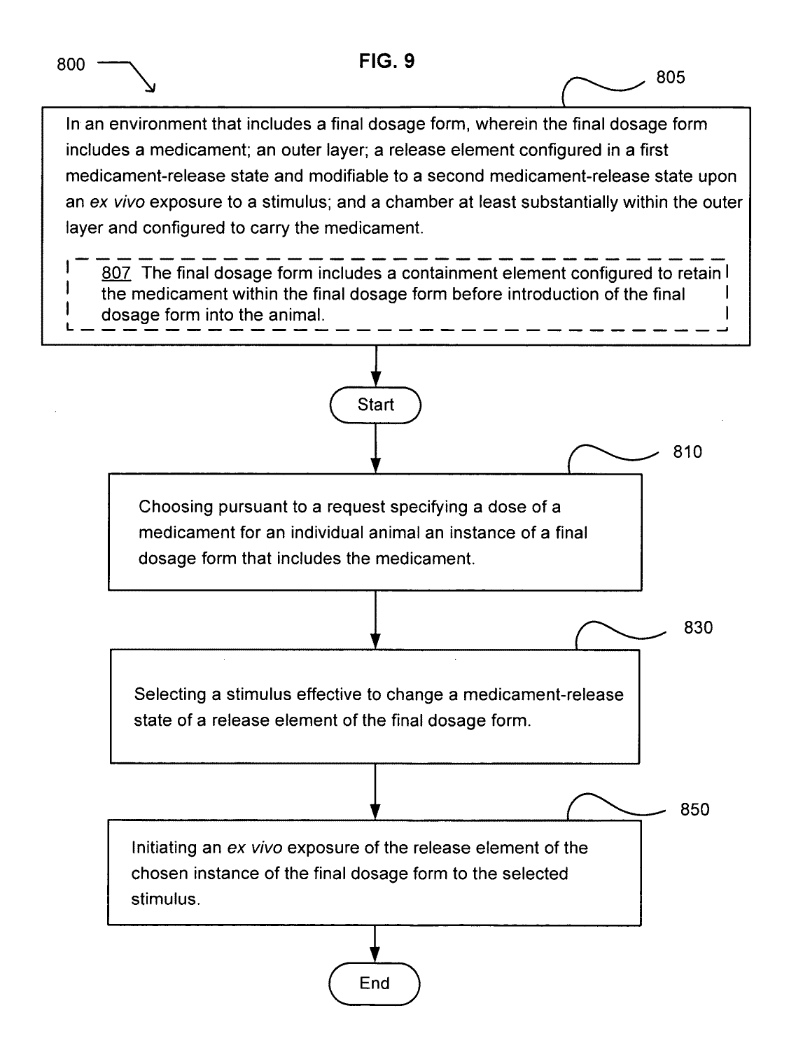

FIG. 9 illustrates an example operational flow 800 fulfilling a request specifying a dose of a medicament for an individual animal. A start operation occurs in an environment that includes a final dosage form. The final dosage form includes an outer layer, a release element configured in a first medicament-release state and changeable to a second medicament-release state upon an ex vivo exposure to a stimulus, a chamber at least substantially within the outer layer and configured to carry the medicament, and the medicament. In an alternative embodiment, the final dosage form further includes a containment element configured to retain the medicament within the final dosage form before introduction of the final dosage form into the animal. After the start operation, the operational flow includes a picking operation 810. The picking operation includes choosing pursuant to the request an instance of a final dosage form that includes the medicament. A decision operation 830 includes selecting a stimulus configured to change a medicament-release state of a release element of the final dosage form. A customization operation 850 includes initiating an ex vivo exposure of the release element of the chosen instance of the final dosage form to the selected stimulus. The operational flow then proceeds to an end operation.

In use of an embodiment, a person such as a pharmacist working in a pharmacy may receive a prescription specifying a dose of a medicament for a patient. A pharmacy typically may have available several different final dosage forms capable of delivering the prescribed medicament dose. For example, the available different dosage forms may include at least one of the embodiments of final dosage forms illustrated in FIGS. 1-5. In a picking operation, the pharmacist chooses pursuant to the request an instance of a final dosage form that includes the medicament. In a decision operation, the pharmacist selects a stimulus effective to change a medicament-release state of a release element of the final dosage form. The pharmacist may select the stimulus after consulting with an instruction presented by at least one of a label on box containing the chosen instance of a final dosage form, a package insert in the box, or an address to electronically published content indicated on the label, or package insert. The pharmacist enters the selected stimulus setting for a stimulus emitter, such as the stimulus emitter 194 of FIG. 1. In a customization operation, the pharmacist initiates an ex vivo exposure of the release element of the chosen instance of the final dosage form to the selected stimulus. The pharmacist may confirm exposure of the release element to the stimulus by referring to the indicator element. For example, the indicator element may change color in response to an exposure to the selected stimulus. If the prescription specifies multiple doses of the medicament for the patient, the pharmacist may repeat the above sequence until sufficient doses have customized. Alternatively, and if appropriate for the chosen final dosage forms, multiple instances of the final dosage form may be ex vivo exposed to the selected stimulus at one time.

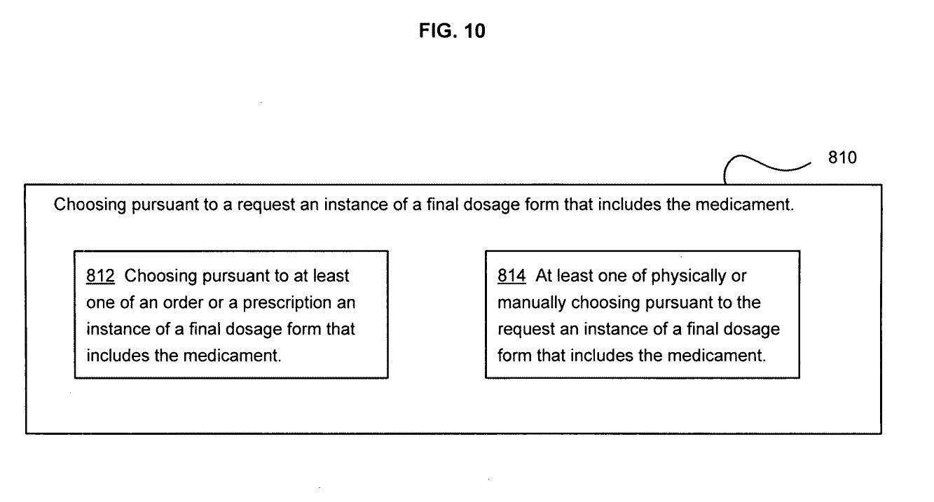

FIG. 10 illustrates an alternative embodiment of the example operational flow 800 of FIG. 9. The picking operation 810 may include at least one additional operation. The at least one additional operation may include an operation 812, or an operation 814. The operation 812 includes choosing pursuant to at least one of an order or a prescription an instance of a final dosage form that includes the medicament. The operation 814 includes at least one of physically or manually choosing pursuant to the request an instance of a final dosage form that includes the medicament.

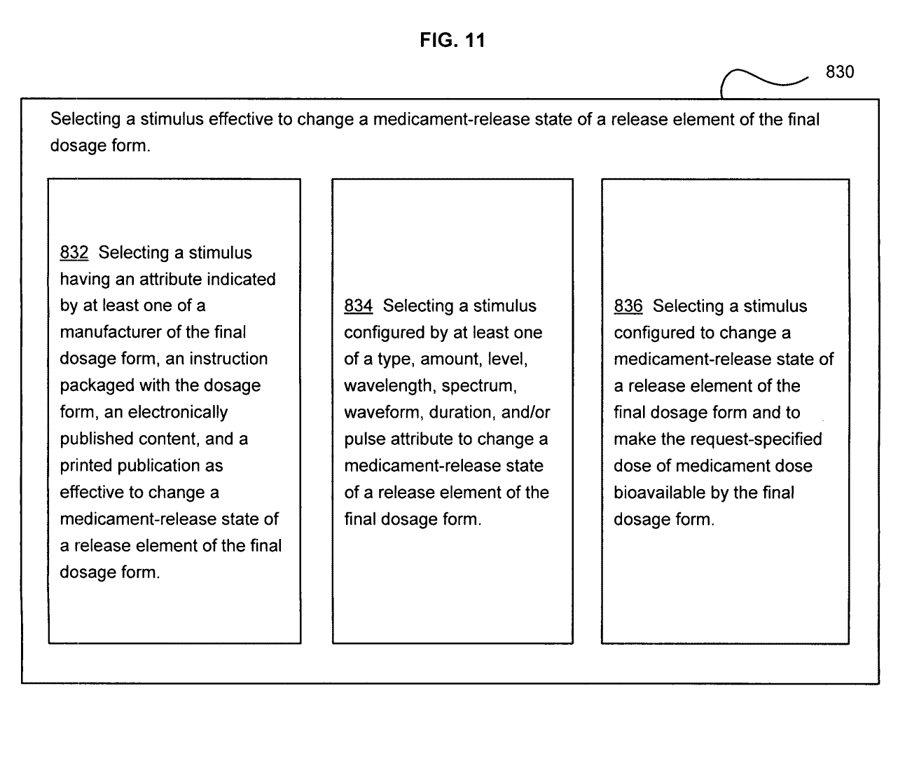

FIG. 11 illustrates another alternative embodiment of the example operational flow 800 of FIG. 9. The decision operation 830 may include at least one additional operation. The at least one additional operation may include an operation 832, an operation 834, or an operation 836. The operation 832 includes selecting a stimulus having an attribute indicated by at least one of a manufacturer of the final dosage form, an instruction packaged with the dosage form, an electronically published content, and a printed publication as effective to change a medicament-release state of a release element of the final dosage form. For example, electronically published content may include a website maintained by the manufacturer of the final dosage form. In a further example, a printed publication may include a reference book, such as Physician's Desk Reference. The operation 834 includes selecting a stimulus configured by at least one of a type, amount, level, wavelength, spectrum, waveform, spatial distribution, duration, or pulse attribute to change a medicament-release state of a release element of the final dosage form. The operation 836 includes selecting a stimulus configured to change a medicament-release state of a release element of the final dosage form and to make the request-specified dose of medicament dose bioavailable by the final dosage form.

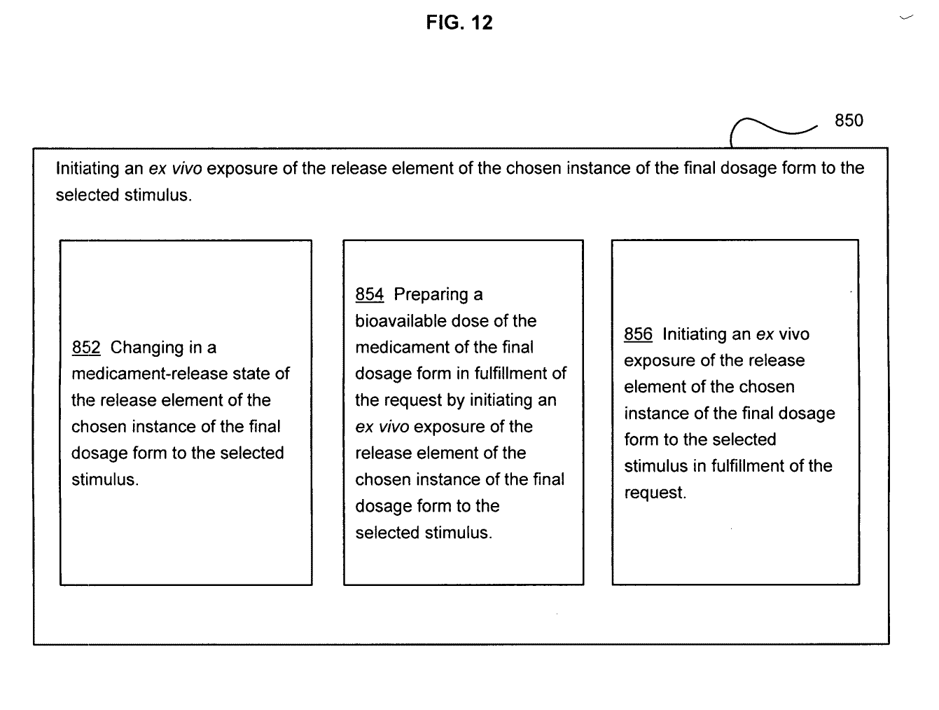

FIG. 12 illustrates an embodiment of the example operation 800 of FIG. 9. The customization operation 850 may include at least one additional operation. The at least one additional operation may include an operation 852, an operation 854, or an operation 856. The operation 852 includes changing a medicament-release state of the release element of the chosen instance of the final dosage form by initiating an ex vivo exposure of the release element of the chosen instance of the final dosage form to the selected stimulus. The operation 854 includes preparing a bioavailable dose of the medicament of the final dosage form in fulfillment of the request by initiating an ex vivo exposure of the release element of the chosen instance of the final dosage form to the selected stimulus. The operation 856 includes initiating an ex vivo exposure of the release element of the chosen instance of the final dosage form to the selected stimulus in fulfillment of the request.

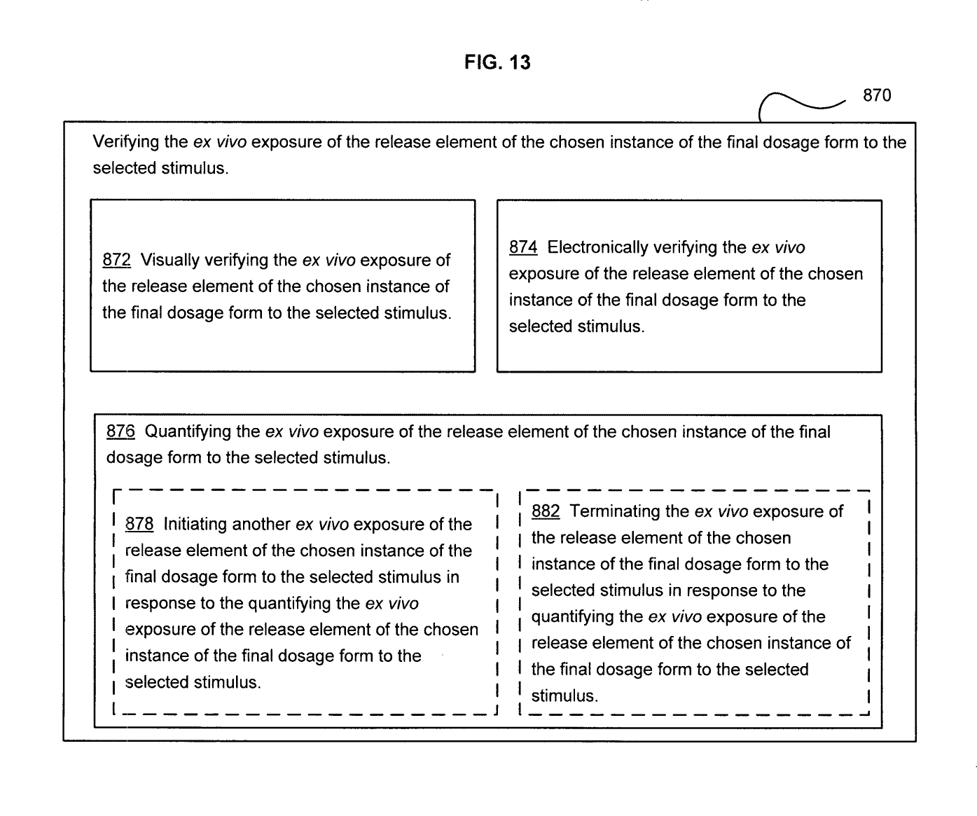

FIG. 13 illustrates an embodiment of the example operational flow 800 of FIG. 9. The operation 870 may include at least one additional operation. The at least one additional operation may include an operation 872, an operation 874, or an operation 876. The operation 870 may include at least one additional operation. The at least one additional operation may include an operation 872, an operation 874, or an operation 876. The operation 872 includes optically verifying the ex vivo exposure of the release element of the chosen instance of the final dosage form to the selected stimulus. For example, optically verifying the ex vivo exposure may be implemented using human vision, machine vision, or ultrasound techniques. The operation 874 includes electronically verifying the ex vivo exposure of the release element of the chosen instance of the final dosage form to the selected stimulus. For example, electronically verifying the ex vivo exposure of the release element may be implemented using a dielectric element having a property that makes an electronically discernable change in response to an exposure to the stimulus. The operation 876 includes quantifying the ex vivo exposure of the release element of the chosen instance of the final dosage form to the selected stimulus. The operation 876 may include at least one additional operation. The at least one additional operation may include an operation 878, or an operation 882. The operation 878 includes initiating another ex vivo exposure of the release element of the chosen instance of the final dosage form to the selected stimulus in response to the quantifying the ex vivo exposure of the release element of the chosen instance of the final dosage form to the selected stimulus. The operation 882 includes terminating the ex vivo exposure of the release element of the chosen instance of the final dosage form to the selected stimulus in response to the quantifying the ex vivo exposure of the release element of the chosen instance of the final dosage form to the selected stimulus.

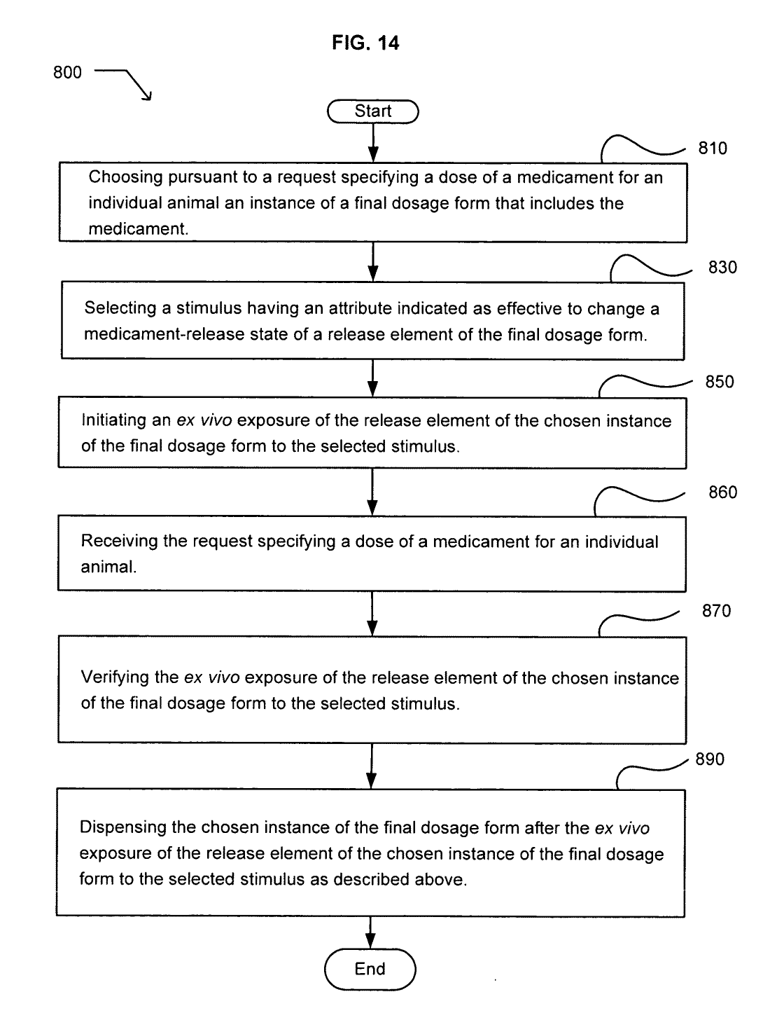

FIG. 14 illustrates an embodiment of the example operational flow 800 of FIG. 9. The operation 800 may include at least one additional operation. The at least one additional operation may include an operation 860, an operation 870, or an operation 890. The operation 860 includes receiving the request specifying a dose of a medicament for an individual animal. The operation 860 may include at least one additional operation. The at least one additional operation may include an operation 862, or an operation 864. The operation 862 (not shown) includes receiving the request specifying an efficacious medicament dose for an individual animal. The operation 864 (not shown) includes receiving the request specifying the final dosage form that includes the medicament for an individual animal.

The operation 870 includes verifying the ex vivo exposure of the release element of the chosen instance of the final dosage form to the selected stimulus. The operation 890 includes dispensing the chosen instance of the final dosage form after the ex vivo exposure of the release element of the chosen instance of the final dosage form to the selected stimulus as described above. The operation 890 may include at least one additional operation, such as an operation 892. The operation 892 (not shown) includes dispensing the ex vivo exposed instance of the final dosage form in a package bearing an identifier of the individual animal. For example, the identifier may include a name, or identification number of the animal.

All references are hereby incorporated by reference herein in their entirety to the extent such subject matter is not inconsistent herewith.

In some embodiments, “configured” includes at least one of designed, set up, shaped, implemented, constructed, or adapted for at least one of a particular purpose, application, or function.

It will be understood that, in general, terms used herein, and especially in the appended claims, are generally intended as “open” terms (e.g., the term “including” should be interpreted as “including but not limited to,” the term “having” should be interpreted as “having at least,” the term “includes” should be interpreted as “includes but is not limited to,” etc.). It will be further understood that if a specific number of an introduced claim recitation is intended, such an intent will be explicitly recited in the claim, and in the absence of such recitation no such intent is present. For example, as an aid to understanding, the following appended claims may contain usage of introductory phrases such as “at least one” or “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a receiver” should typically be interpreted to mean “at least one receiver”); the same holds true for the use of definite articles used to introduce claim recitations. In addition, even if a specific number of an introduced claim recitation is explicitly recited, it will be recognized that such recitation should typically be interpreted to mean at least the recited number (e.g., the bare recitation of “at least two chambers,” or “a plurality of chambers,” without other modifiers, typically means at least two chambers).

Furthermore, in those instances where a phrase such as “at least one of A, B, and C,” “at least one of A, B, or C,” or “an [item] selected from the group consisting of A, B, and C,” is used, in general such a construction is intended to be disjunctive (e.g., any of these phrases would include but not be limited to systems that have A alone, B alone, C alone, A and B together, A and C together, B and C together, or A, B, and C together, and may further include more than one of A, B, or C, such as A₁, A₂, and C together, A, B₁, B₂, C₁, and C₂ together, or B₁ and B₂ together). It will be further understood that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The herein described aspects depict different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality. Any two components capable of being so associated can also be viewed as being “operably couplable” to each other to achieve the desired functionality. Specific examples of operably couplable include but are not limited to physically mateable or physically interacting components or wirelessly interactable or wirelessly interacting components.

While various aspects and embodiments have been disclosed herein, the various aspects and embodiments are for purposes of illustration and are not intended to be limiting, with the true scope and spirit being indicated by the following claims.

What is claimed is:

1. A final dosage form for delivering a medicament to an animal, the final dosage form comprising:
an outer layer;
a release element configured in a first medicament-release state and modifiable to a second medicament-release state upon an ex vivo exposure to a stimulus;
a chamber at least substantially within the outer layer and configured to carry the medicament; and
the medicament.

2. The final dosage form of claim 1, wherein the outer layer includes:
an outer layer of at least one of a tablet, capsule, particle, or solid dosage form.

3. The final dosage form of claim 1, wherein the outer layer includes:
an outer peripheral layer.

4. The final dosage form of claim 1, wherein the outer layer is configured for administration to the animal by at least one of an oral, enteral, inhalation, or implant route.

5. The final dosage form of claim 1, wherein the outer layer includes:
an outer layer configured to release the medicament in an in vivo environment of the animal.

6. The final dosage form of claim 1, wherein the outer layer includes:
an erodible outer layer.

7. The final dosage form of claim 6, wherein the erodible outer layer includes:
an erodible outer layer that is at least one of soluble, permeable, or disintegrable within the animal.

8. The final dosage form of claim 6, wherein the erodible outer layer includes:
an erodible outer layer having at least a portion that is at least one of soluble, permeable, or disintegrable in response to an acidic environment within the animal.

9. The final dosage form of claim 6, wherein the erodible outer layer includes:
an erodible outer layer having at least a portion that is at least one of soluble, permeable, or disintegrable in response to a basic environment within the animal.

10. The final dosage form of claim 1, wherein the outer layer includes:
an outer portion of a molecule.

11. The final dosage form of claim 1, wherein the outer layer includes:
an outer portion of a particle.

12. The final dosage form of claim 1, wherein the outer layer includes:
an outer layer configured to allow an in vivo discharge of at least a portion of the medicament from the chamber after an exposure of the release element to the stimulus.

13. The final dosage form of claim 1, wherein the outer layer includes:
an outer layer configured in cooperation with the release element to allow an in vivo discharge of at least a portion of the medicament from the chamber after an exposure of the release element to the stimulus.

14. The final dosage form of claim 1, wherein the outer layer includes:
an outer layer that includes at least a portion of the release element.

15. The final dosage form of claim 1, wherein the outer layer includes:
an outer layer configured to contain the medicament until the final dosage form is administered into the animal.

16. The final dosage form of claim 1, wherein the release element configured in a first medicament-release state and modifiable to a second medicament-release state upon an ex vivo exposure to a stimulus includes:
a release element disposed at least partially within the outer layer and configured in a first medicament-release state and modifiable to a second medicament-release state upon an ex vivo exposure to a stimulus.

17. The final dosage form of claim 1, wherein the release element configured in a first medicament-release state and modifiable to a second medicament-release state upon an ex vivo exposure to a stimulus includes:
a release element configured in a first medicament-release state and reconfigurable to a second medicament-release state upon an ex vivo exposure to a stimulus.

18. The final dosage form of claim 1, wherein the release element configured in a first medicament-release state and modifiable to a second medicament-release state upon an ex vivo exposure to a stimulus includes:
a release element configured in a first medicament-release state and changeable to a second medicament-release state upon an ex vivo exposure to a stimulus.

19. The final dosage form of claim 1, wherein the release element configured in a first medicament-release state and modifiable to a second medicament-release state upon an ex vivo exposure to a stimulus includes:
a release element configured in a first medicament-release state and modifiable to a second medicament-release state upon an ex vivo exposure to a non-ionizing radiation.

20. The final dosage form of claim 1, wherein the release element configured in a first medicament-release state and modifiable to a second medicament-release state upon an ex vivo exposure to a stimulus includes:
a release element configured in a first medicament-release state and modifiable to a second medicament-release state upon an ex vivo exposure to an electromagnetic radiation.

21. The final dosage form of claim 1, wherein the release element configured in a first medicament-release state and modifiable to a second medicament-release state upon an ex vivo exposure to a stimulus includes:
a release element configured in a first medicament-release state and modifiable to a second medicament-release state upon an ex vivo exposure to at least one of a light radiation, terahertz radiation, microwave radiation, and radio wave radiation.

22. The final dosage form of claim 1, wherein the release element configured in a first medicament-release state and modifiable to a second medicament-release state upon an ex vivo exposure to a stimulus includes:
a release element configured in a first medicament-release state and modifiable to a second medicament-release state upon an ex vivo exposure to a magnetic stimulus.

23. The final dosage form of claim 1, wherein the release element configured in a first medicament-release state and modifiable to a second medicament-release state upon an ex vivo exposure to a stimulus includes:
a release element configured in a first medicament-release state and modifiable to a second medicament-release state upon an ex vivo exposure to an electric field stimulus.

24. The final dosage form of claim 1, wherein the release element configured in a first medicament-release state and modifiable to a second medicament-release state upon an ex vivo exposure to a stimulus includes:
a release element configured in a first medicament-release state and modifiable to a second medicament-release state upon an ex vivo exposure to an energetic stimulus.

25. The final dosage form of claim 1, wherein the release element configured in a first medicament-release state and modifiable to a second medicament-release state upon an ex vivo exposure to a stimulus includes:
a release element configured in a first medicament-release state and modifiable to a second medicament-release state upon an ex vivo exposure to a chemical stimulus.

26. The final dosage form of claim 1, wherein the release element configured in a first medicament-release state and modifiable to a second medicament-release state upon an ex vivo exposure to a stimulus includes:
a release element configured in a first medicament-release state and modifiable to a second medicament-release state upon an ex vivo exposure to a mechanical stimulus.

27. The final dosage form of claim 1, wherein the release element configured in a first medicament-release state and modifiable to a second medicament-release state upon an ex vivo exposure to a stimulus includes:
release element configured in a first medicament-release state and modifiable to a second medicament-release state upon an ex vivo exposure to a stimulus, the release element including at least one of a mechanically activatable structure, heat activatable structure, or pressure activatable structure.

28. The final dosage form of claim 1, wherein the release element configured in a first medicament-release state and modifiable to a second medicament-release state upon an ex vivo exposure to a stimulus includes:
a release element configured in a first medicament-release state and modifiable to a second medicament-release state upon an ex vivo exposure to at least one of an activation stimulus, or an actuation stimulus.

29. The final dosage form of claim 1, wherein the release element configured in a first medicament-release state and modifiable to a second medicament-release state upon an ex vivo exposure to a stimulus includes:
a release element configured in a first medicament-release state and modifiable to a second medicament-release state upon an ex vivo exposure to a de-activation stimulus.

30. The final dosage form of claim 1, wherein the release element configured in a first medicament-release state and modifiable to a second medicament-release state upon an ex vivo exposure to a stimulus includes:
release element configured in a first medicament-release state and modifiable to a second medicament-release state upon an ex vivo exposure to at least one of at least one of a thermal, acoustic, and ultrasound stimulus.

31. The final dosage form of claim 1, wherein the first medicament-release state is configured to retard medicament release in vivo and the second medicament-release state is configured to allow medicament release in vivo.

32. The final dosage form of claim 1, wherein the first medicament-release state is configured to allow medicament release in vivo and the second medicament-release state is configured to retard medicament release in vivo.

33. The final dosage form of claim 1, wherein the release element includes at least one of polymeric micelle, nanoparticles, cyclodextrin, gel, gel matrix, hydrogel, or cellulose.

34. The final dosage form of claim 1, wherein the release element includes at least one of a poly(vinyl alcohol), gel, gel matrix, hydrogel, or azopolymer membrane.

35. The final dosage form of claim 1, wherein the release element includes at least one of a foil, gold foil, a liposome, wax, or dielectric/wax composite.

36. The final dosage form of claim 1, wherein the release element includes at least one of polyanhidrides, polyglycolides, polyactides, poly(vinyl acetate), poly(glycolic acid), poly (ethylene), poly(lactic acid), or chitosan.

37. The final dosage form of claim 1, wherein the release element configured in a first medicament-release state and modifiable to a second medicament-release state upon an ex vivo exposure to a stimulus includes:
a release element enclosing the chamber, configured in a first medicament-release state, and modifiable to a second medicament-release state upon an ex vivo exposure to a stimulus.

38. The final dosage form of claim 1, wherein the release element configured in a first medicament-release state and modifiable to a second medicament-release state upon an ex vivo exposure to a stimulus includes:
a release element encapsulating the chamber, configured in a first medicament-release state, and modifiable to a second medicament-release state upon an ex vivo exposure to a stimulus.

39. The final dosage form of claim 1, wherein the release element configured in a first medicament-release state and modifiable to a second medicament-release state upon an ex vivo exposure to a stimulus includes:
a release element encapsulating the medicament in cooperation with the chamber, configured in a first medicament-release state, and modifiable to a second medicament-release state upon an ex vivo exposure to a stimulus.

40. The final dosage form of claim 1, wherein the release element configured in a first medicament-release state and modifiable to a second medicament-release state upon an ex vivo exposure to a stimulus includes:
a release element obstructing an aperture of the chamber, configured in a first medicament-release state, and modifiable to a second medicament-release state upon an ex vivo exposure to a stimulus.

41. The final dosage form of claim 1, wherein the release element configured in a first medicament-release state and modifiable to a second medicament-release state upon an ex vivo exposure to a stimulus includes:
at least two particles each respectively forming a chamber carrying a respective instance of the medicament, the at least two particles configured in a first medicament-release state, and modifiable to a second medicament-release state upon an ex vivo exposure of the at least two particles to a stimulus.

42. The final dosage form of claim 1, wherein the release element configured in a first medicament-release state and modifiable to a second medicament-release state upon an ex vivo exposure to a stimulus includes:
a release element configured in a first medicament-release state and modifiable to a second medicament-release state upon an ex vivo exposure to a stimulus, and configured to contain the medicament at least until the final dosage form is administered into the animal.

43. The final dosage form of claim 1, wherein the chamber at least substantially within the outer layer and configured to carry the medicament further includes:
a singular chamber structure at least substantially within the outer layer and configured to carry the medicament.

44. The final dosage form of claim 1, wherein the chamber at least substantially within the outer layer and configured to carry the medicament further includes:
a chamber at least substantially within the outer layer and configured to carry the medicament, the chamber including at least one of an aggregation of cells formed by interstitial cavities, at least two smaller chambers, interstices of a molecular structure, or amorphous cavities.

45. The final dosage form of claim 1, wherein the chamber at least substantially within the outer layer and configured to carry the medicament includes:
a chamber disposed within the outer layer and configured to carry the medicament.

46. The final dosage form of claim 1, wherein the chamber at least substantially within the outer layer and configured to carry the medicament includes:
a chamber at least substantially within the outer layer and configured to carry the medicament until released by the release element.

47. The final dosage form of claim 1, wherein the chamber at least substantially within the outer layer and configured to carry the medicament includes:
at least one chamber at least substantially within the outer layer and configured to carry the medicament.

48. The final dosage form of claim 1, wherein the chamber at least substantially within the outer layer and configured to carry the medicament includes:
at least two chambers configured to carry respective instances of the medicament.

49. The final dosage form of claim 1, wherein the chamber at least substantially within the outer layer and configured to carry the medicament includes:
a first chamber configured to carry a first medicament and a second chamber configured to carry a second medicament.

50. The final dosage form of claim 1, wherein the chamber at least substantially within the outer layer and configured to carry the medicament includes:
a chamber configured to confine the medicament in cooperation with the release element.

51. The final dosage form of claim 1, wherein the chamber at least substantially within the outer layer and configured to carry the medicament includes:
least one chamber configured to confine the medicament in a structural cooperation with the release element.

52. The final dosage form of claim 1, wherein the chamber at least substantially within the outer layer and configured to carry the medicament includes:
a chamber configured to carry the medicament, and configured to release at least a portion of the medicament from the outer layer upon at least one of a bursting, puncture, permeation, dissolution, and disintegration of the release element.

53. The final dosage form of claim 1, wherein the chamber at least substantially within the outer layer and configured to carry the medicament includes:
a first chamber configured to carry a first constituent of the medicament and a second chamber configured to carry a second constituent of the medicament.

54. The final dosage form of claim 1, wherein the chamber at least substantially within the outer layer and configured to carry the medicament includes:
a first chamber configured to carry a first reactant of the medicament and a second chamber configured to carry a second reactant of the medicament.
